(19) United States
(12) Patent Application Publication
(10) Pub. No.: US 2005/0096602 A1
(43) Pub. Date: May 5, 2005

(54) Plunger structure for syringes

(76) Inventor: Hui-Chiang Lin, Chunghua Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/947,150

(22) Filed: Sep. 23, 2004

(30) Foreign Application Priority Data
Nov. 4, 2003 (CN) 2003201026434

(51) Int. Cl. A61M 5/315

(52) U.S. Cl. 604/221

(57) ABSTRACT

A syringe includes barrel with a plunger inserted in the barrel and a plurality of flexible stubs extending inclinedly from an inner periphery of an opening in the first end of the barrel. A cannula is connected to the first end of the barrel. The plunger includes a tip portion which is connected to an end of the plunger and includes an enlarged portion which moves through a space enclosed by the flexible stubs when the plunger is moved to its limit position in the barrel and the tips of the flexible stubs engaged with an end of the enlarged portion so as to prevent the plunger from being pulled backward. The end of the plunger that the tip portion is connected includes a plurality of breaking slits and the tip portion can be easily separated from the plunger after use.

### FIELD OF THE INVENTION

The present invention relates to a hypodermic syringe whose plunger can be broken after use.

### BACKGROUND OF THE INVENTION

A conventional hypodermic syringe generally includes a barrel with a locking tip on a first end thereof and a needle cannula is fixed to the locking tip. A plunger is movably received in the barrel so as to push the medicine in the barrel from the needle cannula. A protection cap is used to mount to the locking tip so as to receive the needle cannula in the cap. It will be a serious concern that the needle cannula could hurt the medical personnel during removing the protection cap from the locking tip before proceeding syringing and/or re-mounting the protection cap to the locking tip after the syringing process is finished. Besides, the discarded syringe could be mis-used by the those people who utilize the syringes, no matter whatever condition the syringes is, to inject drug into their bodies.

The present invention intends to provide a syringe wherein the plunger can be broken and separated from the cannula after use.

### SUMMARY OF THE INVENTION

The present invention relates to syringe which comprises a barrel with a passage defined therein and a plunger is inserted in the passage. A connection end extends from a first end of the barrel and includes an opening co-axially communicating with the passage and a plurality of flexible stubs extend inclinedly from an inner periphery of the opening. A locking tip is mounted to the connection end and has an extension portion on which a cannula is mounted. A through hole is defined through the locking tip. The plunger has a tip portion at an end of the plunger and a stopper is mounted to the tip portion. A plurality of breaking slits are defined in the end on which the tip portion is connected. The tip portion includes a first enlarged portion extending radially therefrom which push the flexible stubs outward and moves through an space enclosed by tips of the flexible stubs till the free ends of the flexible stubs are engaged with an end of the first enlarged portion so as to prevent the plunger from being pulled backward.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the syringe of the present invention;

FIGS. 2A to 2D show the continuous movements of the plunger being pushed toward the first end of the barrel;

FIG. 3A shows that the plunger is pushed to its limit position and the user shake the plunger;

FIG. 3b shows the plunger is separated from the tip portion;

FIG. 4 shows the flexible stubs are made with an independent member;

FIG. 5 is an exploded view to show another embodiment of the syringe of the present invention;

FIG. 6A shows the flange on the plunger pushes the flexible stubs;

FIG. 6B shows the plunger is moved to its limit position and the user shake the plunger, and

FIG. 6C shows that the end section is separated from of the plunger.

### DETAILED DESCRIPTION OF THE PREFERRED EMBODIMENT

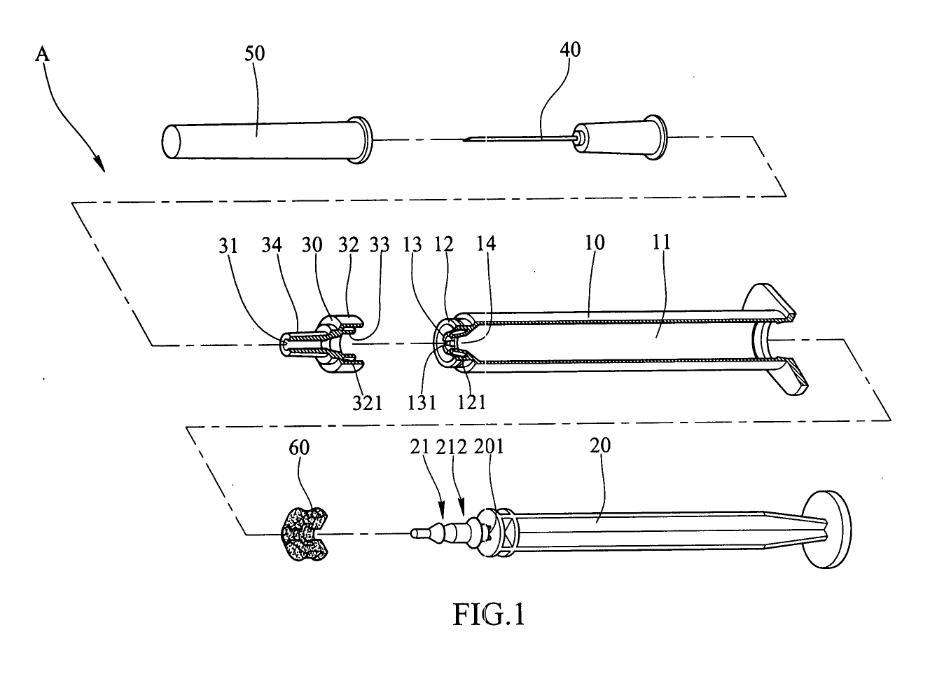

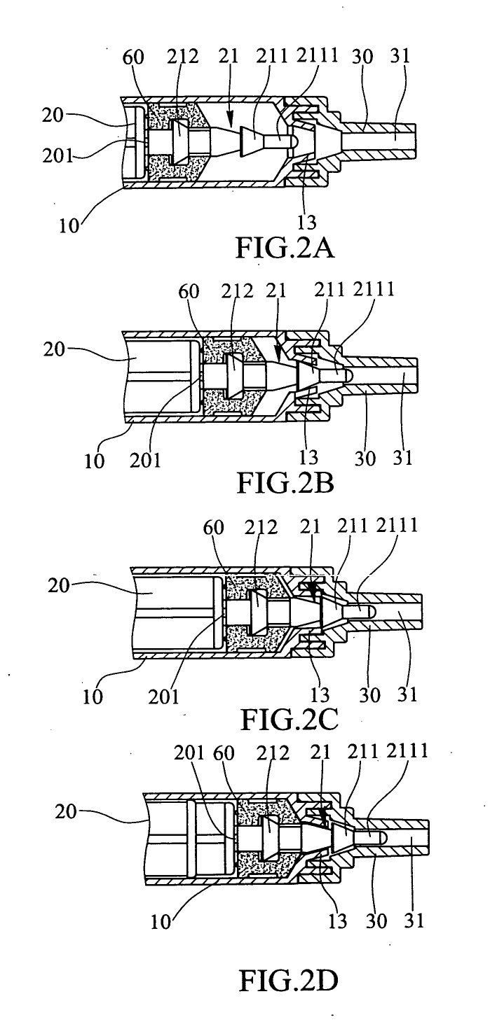

Referring to FIGS. 1 and 2A, the syringe “A” of the present invention comprises a barrel 10 with a passage 11 defined therein and a connection end 12 extends from a first end of the barrel 10. The connection end 12 includes an opening 14 co-axially communicating with the passage 13 and a plurality of flexible stubs 13 extend inclinedly from an inner periphery of the opening 14. The flexible stubs 13 are separated by gaps 131 such that the flexible stubs 13 enclose a tapered space and can be pushed outward. A groove 121 is defined between the flexible stubs 13 and an inner periphery of the connection end 12. A locking tip 30 is mounted to the connection end 12 and has a skirt portion 32 mounted to an outer periphery of the connection end 12 and an annular lip 33 inserted in the groove 121. An extension portion 34 extends from the locking tip 30 and a cannula 40 is mounted to the extension portion 34. A through hole 31 is defined through the locking tip 30.

A plunger 20 is movably inserted in the passage 14 in the barrel 10 from a second end of the barrel 10 and has a tip portion 21 is connected to at an end of the plunger 20. The tip portion 21 includes a first enlarged portion 211 and a second enlarged portion 212 extending radially therefrom. The second enlarged portion 212 includes a tapered outer periphery. A stopper 60 is mounted to the tip portion 21 and includes a mounting passage and an enlarged space extends outward from an inner periphery of the mounting passage. The second enlarged portion 212 is received in the enlarged space.

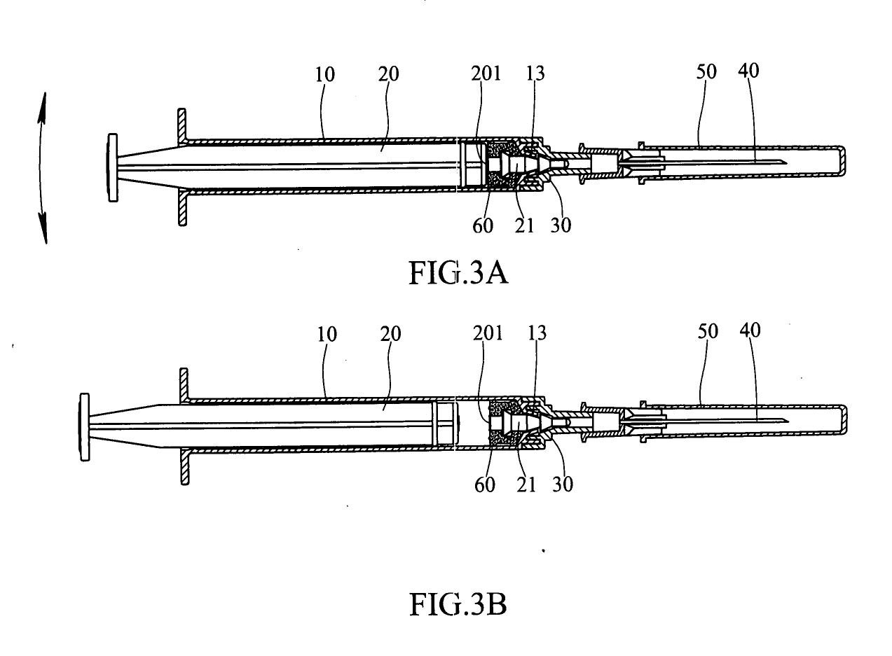

Referring to FIGS. 2B, 2C and 2D, when pushing the plunger 20, the first enlarged portion 211 of the tip portion 21 pushes the flexible stubs 13 outward and moves through the space enclosed by tips of the flexible stubs 13, till free ends of the flexible stubs 13 are engaged with an end of the first enlarged portion 211, and this is the limit position of the plunger 20. A leading tip 2111 of the tip portion 21 then seals the through hole 31 of the locking tip 30. The engagement of the free ends of the flexible stubs 13 and the first enlarged portion 211 prevents the plunger 20 from being pulled backward. It is noted that a plurality of breaking slits 201 are defined in the end on which the tip portion 21 is connected. Referring to FIGS. 3A and 3B, the user then shanks the plunger 20 and the tip portion 21 is separated from the plunger 20, by this way, The syringe cannot be reused again. A cap 50 is then mounted to the cannula 40.

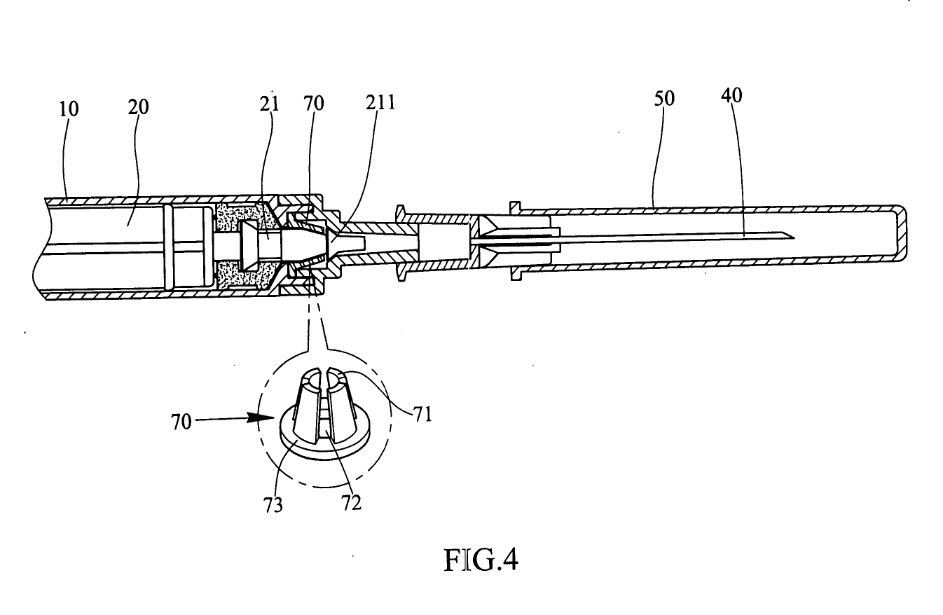

FIG. 4 shows that the flexible stubs are made with an independent member 70 which includes a ring 73 with a plurality of flexible stubs 71 extending from an end thereof. Gaps 72 are defined between the flexible stubs 71 such that the flexible stubs 71 can be pushed outward. The independent member 70 is securely mounted onto the tip portion 21 and the plunger 20 can be pushed to its limit position where the first enlarged portion 211 is engaged with free ends of the flexible stubs 71 to prevent the plunger 20 from being pulled backward.

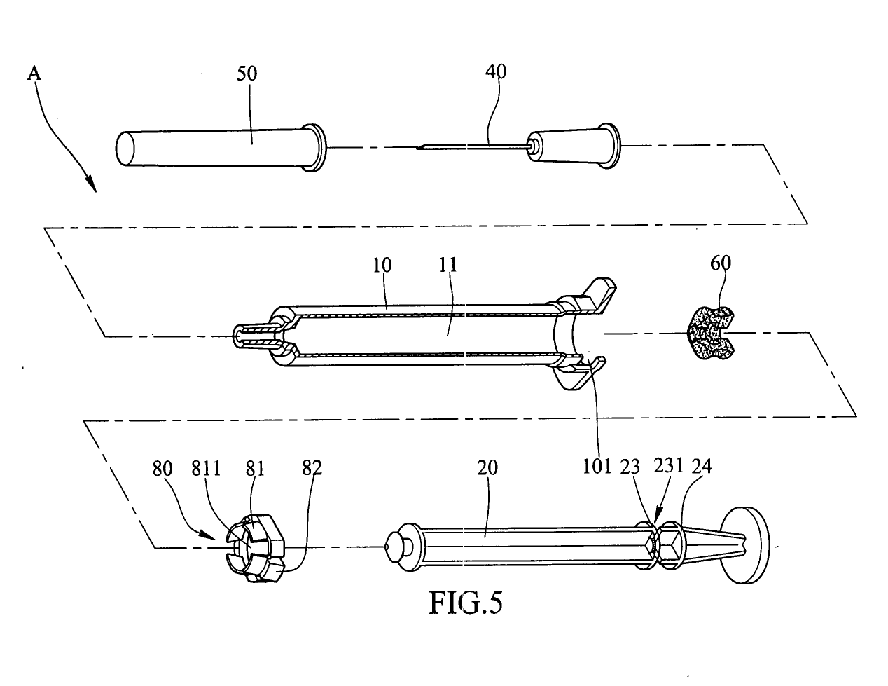

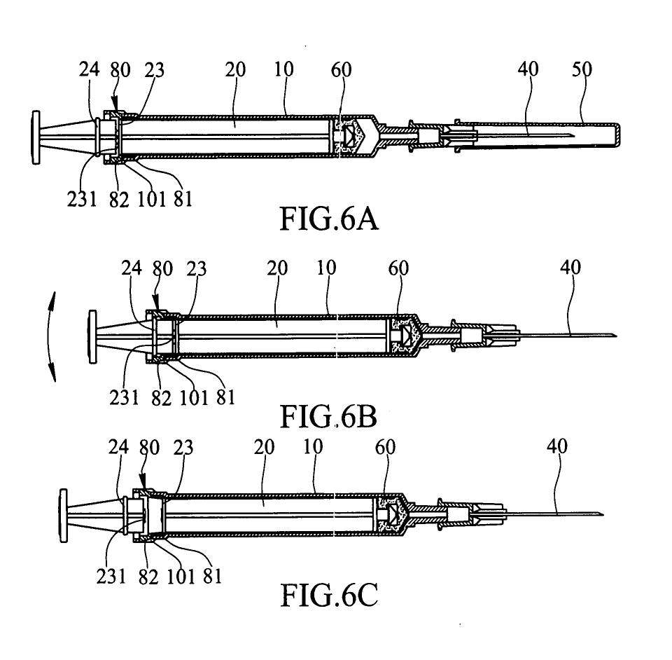

Referring to FIGS. 5, 6A, 6B and 6C, the syringe comprises a barrel 10 with a passage 11 defined therein and a cannula 40 is mounted to a first end of the barrel 10. A second end of the barrel 10 is an open end through which a plunger 20 is inserted. A notch 101 is defined in an inner periphery of the second end of the barrel 10. A ring 80 is engaged with the open end of the barrel 10 by engaging a protrusion 82 of the ring 80 with the notch 101. A plurality of flexible stubs 13 extend inclinedly from an inner periphery of the ring 80.

A stopper 60 is connected to a front end of the plunger 20 and an end section 24 is connected to a second end of the plunger 20. A flange 23 extends radially from the second end of the plunger 20. A plurality of breaking slits 231 are defined in the second end of the plunger 20 where the end section 24 is connected such that the end section 24 can be easily separated from the plunger 20.

When pushing the plunger 20 toward the cannular 40, the flange 23 pushes the flexible stubs 81 outward till the free ends of the flexible stubs 81 are engaged with an end of the flange 23 so as to prevent the plunger 20 from pulling backward. The length of the plunger 20 is set such that the stopper 60 is moved to an inner end of the first end of the barrel 10 when the free ends of the flexible stubs 81 are engaged with an end of the flange 23. The end section 24 can be separated from the plunger 20 by shaking the end section 24. A cap 50 is mounted to the cannula 40.

While we have shown and described the embodiment in accordance with the present invention, it should be clear to those skilled in the art that further embodiments may be made without departing from the scope of the present invention.

1. A syringe comprising:
a barrel with a passage defined therein and a connection end extending from a first end of the barrel, the connection end including an opening co-axially communicating with the passage and a plurality of flexible stubs extending inclinedly from an inner periphery of the opening;
a locking tip mounted to the connection end and having an extension portion on which a cannula is mounted, a through hole defined through the locking tip, and
a plunger movably inserted in the passage in the barrel from a second end of the barrel and having a tip portion connected to at an end of the plunger, a stopper mounted to the tip portion, the tip portion including a first enlarged portion extending radially therefrom which push the flexible stubs outward and moves through an space enclosed by free ends of the flexible stubs, the free ends of the flexible stubs engaged with an end of the first enlarged portion so as to prevent the plunger from being pulled backward.

2. The syringe as claimed in claim 1, wherein a plurality of breaking slits are defined in the end on which the tip portion is connected such that the tip portion is separated from the plunger.

3. The syringe as claimed in claim 1, wherein a groove is defined between the flexible stubs and an inner periphery of the connection end, the locking tip having a skirt portion mounted to an outer periphery of the connection end and an annular lip inserted in the groove.

4. The syringe as claimed in claim 1, wherein the tip portion includes a second enlarged portion which includes a tapered outer periphery, the stopper includes a mounting passage and an enlarged space extends outward from an inner periphery of the mounting passage, the stopper is mounted to the tip portion and the second enlarged portion is received in the enlarged space.

5. The syringe as claimed in claim 1, wherein the tip portion includes a leading tip which is sized to seal the through hole of the locking tip.

6. A syringe comprising:
a barrel with a passage defined therein and a cannula mounted to a first end of the barrel, a second end of the barrel being an open end;
a ring engaged with the open end of the barrel and including a plurality of flexible stubs extending inclinedly from an inner periphery of the ring, and
a plunger movably inserted in the passage in the barrel from the second end of the barrel and a stopper connected to a front end of the plunger, an end section connected to a second end of the plunger and a flange extending radially from the second end of the plunger, free ends of the flexible stubs engaged with an end of the flange so as to prevent the plunger from being pulled backward when the stopper is moved to an inner end of the first end of the barrel.

7. The syringe as claimed in claim 6, wherein a plurality of breaking slits are defined in the second end of the plunger where the end section is connected such that the end section is separated from the plunger.

8. The syringe as claimed in claim 6, wherein a notch is defined in an inner periphery of the second end of the barrel and the ring includes a protrusion which is engaged with the notch.
